(19) United States
(12) Patent Application Publication
(10) Pub. No.: US 2006/0004251 A1
(43) Pub. Date: Jan. 5, 2006

(54) Phallic device mount with rotary switch actuator

(76) Inventors: Dale Bookwalter, Philomath, OR (US); Rhonda Bookwalter, Philomath, OR (US)

Correspondence Address: IPSOLON LLP, 805 SW BROADWAY, #2740, PORTLAND, OR, 97205, US

(21) Appl. No.: 11/049,593

(22) Filed: Feb. 1, 2005

Related U.S. Application Data
(63) Continuation-in-part of application No. 10/850,287, filed on May 19, 2004.
(60) Provisional application No. 60/541,030, filed on Feb. 1, 2004.

(51) Int. Cl. A61F 5/00

(52) U.S. Cl. 600/38

(57) ABSTRACT

A phallic device mount for operably securing a conventional hand-held phallic device thereto while still allowing activation of a rotary switch on the phallic device is disclosed. In one embodiment, a ring is operably secured to the rotary switch and slidably received within a recess on the mount to define a pivot axis, thereby allowing a user to adjust the rotary switch by turning the ring about the pivot axis. Accordingly, the mount provides hands-free use of all available features of the phallic device.

### CROSS-REFERENCE TO RELATED APPLICATIONS

This application is a continuation-in-part of U.S. patent application Ser. No. 10/850,287 filed on May 19, 2004. This application also claims priority to U.S. provisional patent application Ser. No. 60/532,501, filed on Dec. 24, 2003, and 60/541,030, filed on Feb. 1, 2004.

### TECHNICAL FIELD

This invention relates to mounts to facilitate use of phallic devices such as penis substitutes, elongate sexual devices, dildos, vibrators, and the like.

### BACKGROUND OF THE INVENTION

Phallic devices have been used for many years primarily for sexual pleasure both by couples and by people who do not have a sex partner. The phallic device is an elongate object sized and shaped to be inserted into the vagina of a woman and/or the anus of a person. Usually, phallic devices are either a smooth edged contoured cylinder or they are shaped to resemble either an erect or flaccid human penis. More recently, modern fabrication methods and materials allow phallic devices to be formed into any shape, size, or texture.

The use of phallic devices for obtaining sexual pleasure is gaining popularity along with social acceptance. This increased popularity is due to a number of social and environmental factors including: 1) an increase in sexually transmitted diseases such as AIDS and the like; 2) an increasingly open attitude towards human sexuality within our society; and 3) a desire for couples and people who do not have a sex partner to privately and safely explore a wide variety of sexual experiences.

Despite increasing social acceptance of phallic devices, most users prefer to keep them, and any related structures associated with using them, concealed when not in use.

Historically, most phallic devices are held by the user themselves or by a sexual partner during use. Unfortunately, the number of sexual activities that may be simulated or performed by a user is necessarily limited by the phallic device holders' reach and the holder's range of movement available. Moreover, physically challenged individuals may not have the capacity to properly hold the phallic device for use. Accordingly, their ability to enjoy the benefits of these devices is limited.

Recently, efforts have been made to provide hands-free use of phallic devices. For example, U.S. Pat. No. 5,690,604 to Barnett (“Barnett”) discloses mounting a phallic device to a C-shaped clamp. The C-shaped clamp may then be secured by a user to a support structure, such as a bedpost or the like. Similarly, U.S. Pat. No. 5,127,396 to McAllister (“McAllister”) discloses mounting a phallic device to a universal mount. The universal mount may then be strapped around a wearer's torso, thereby allowing the wearer to insert the phallic device into their partner and simulate thrusting movements normally associated with performing the sexual act.

Despite the benefits offered by these types of phallic device mounting structures, they have several drawbacks. For example, a hand-tightened clamp on a bed frame is often insufficient to withstand the forces generated during use of the phallic device. Accordingly, the phallic device may tend to move or detach from the bed frame during use. Such movement can damage the bed frame, or worse yet, injure a user.

Moreover, the mounting structure in Barnett requires a user to have a suitably sized support structure for engaging the C-shaped clamp positioned at substantially the same height where the phallic device must be positioned for use. In practice, this positioning for the support structure is difficult to find in a suitably private environment sufficient for using the phallic device therein.

Similarly, while the strap on mounting structure in McAllister may offer sexual partners an opportunity to engage in a new sexual experience together, it does not allow hands free use of the phallic device by a single user. Also, both the C-shaped clamp in Barnett and the strap on mounting structure in McAllister are not particularly aesthetically pleasing, nor do they allow for easy concealment and storage when not in use.

In addition, rigidly mounted phallic devices require the user to physically move themselves about the phallic device, usually by standing or kneeling adjacent to the phallic device and thrusting themselves against the phallic device. Such actions can be awkward and/or painful. Moreover, physically challenged individuals my not have the physical ability to easily perform these types of maneuvers.

Also, most known phallic device mounts do not allow a user to detachably secure a common hand-held vibrator or the like thereto, much less providing easy activation of any rotary switches on such devices.

### SUMMARY OF THE INVENTION

Accordingly, despite the available phallic device mounting structures, there remains a need for phallic device mount that allows a user to mount a common hand-held phallic device thereto so as to provide hands-free use of the phallic device, that allows easy activation of any activation or adjustment switches attached to the phallic device, that is easy to assemble, adjust and use, and that provides rigid support for the phallic device during use, and that may be easily concealed and stored when not in use.

More preferably, the phallic device is detachably secured to the mount so as to extend substantially horizontally above a substantially horizontal surface. Preferably, the phallic device is a common hand-held elongate vibrator with a rotary activation switch operably secured at one distal end. A resilient phallic device shaft mount is secured to the shaft of the phallic device and then detachably secured within a slot of the mount. The rotary switch of the phallic device is snuggly received within a ring that is inserted within a recess in the mount between a planar surface and the mount thereby allowing the rotary switch to be activated while the phallic device is secured within the mount simply by moving the right either clockwise or counterclockwise as needed.

In one disclosed embodiment, a dolly sized to support a user lying on their back slides substantially along the horizontal surface thereby allowing the user to use the phallic device by sliding themselves on the dolly repeatedly toward and away from the phallic device. Even more preferably, the dolly is biased to a neutral position and operably secured to one or more rails thereby maintaining proper alignment during use and offering a pleasing returning force urging the user toward the phallic device during use.

In addition to other benefits that will become apparent in the following disclosure, the present invention fulfills these needs.

### BRIEF DESCRIPTION OF THE DRAWINGS

FIG. 1 is an isometric, front view of a phallic device mount in accordance with an embodiment of the present invention showing a possible in-use position and a possible orientation with respect to a bed.

FIG. 2 is an isometric, back view of the phallic device mount structure of FIG. 1 showing a possible retracted position.

FIG. 3 is an exploded, isometric view of the phallic device mount of FIG. 1.

FIG. 4 is a cross sectional view of the phallic device mount of FIG. 1 taken along line 4-4 of FIG. 1.

FIG. 5 is an isometric, front view of a phallic device mount in accordance with an alternative embodiment of the present invention showing a possible in-use position and a possible orientation with respect to a bed.

FIG. 6 is an isometric, back view of the phallic device mount structure of FIG. 5 showing possible movement of a bed-mounting portion.

FIG. 7 is a cross sectional view of the phallic device mount of FIG. 5 taken along line 7-7 of FIG. 6.

FIG. 8 is an isometric view of a phallic device mounting portion of an embodiment of the present invention used in a possible alternative mounting to an auxiliary support structure in accordance with an embodiment of the present invention.

FIG. 9 is a section view of the phallic device-mounting portion of FIG. 8 taken along line 9-9 of FIG. 8.

FIG. 10 is an isometric view of the phallic device mount of FIG. 1 positioned adjacent to a dolly in accordance with an embodiment of the present invention showing a possible neutral position of the dolly relative to the phallic device mount.

FIG. 11 is an exploded view of the dolly of FIG. 10 in the possible neutral position.

FIG. 12 is a top view of the unassembled components forming an embodiment of the phallic device mount with a commercially available hand-held vibrator having a rotary activation switch thereon.

FIG. 13 is an enlarged isometric view of the components of FIG. 13 showing a possible assembled view in accordance with an embodiment of the present invention.

FIG. 14 is a lower, isometric view of the assembled components of FIG. 13.

FIG. 15 is a lower, isometric view showing a possible installation of ring 412 into recess 60.

FIG. 16 is a lower, isometric view showing possible installation of the phallic device 22′ secured to the ring 412 with the phallic device shaft mount 404 positioned for installation on the shaft 408 of the phallic device 22′.

### DETAILED DESCRIPTION

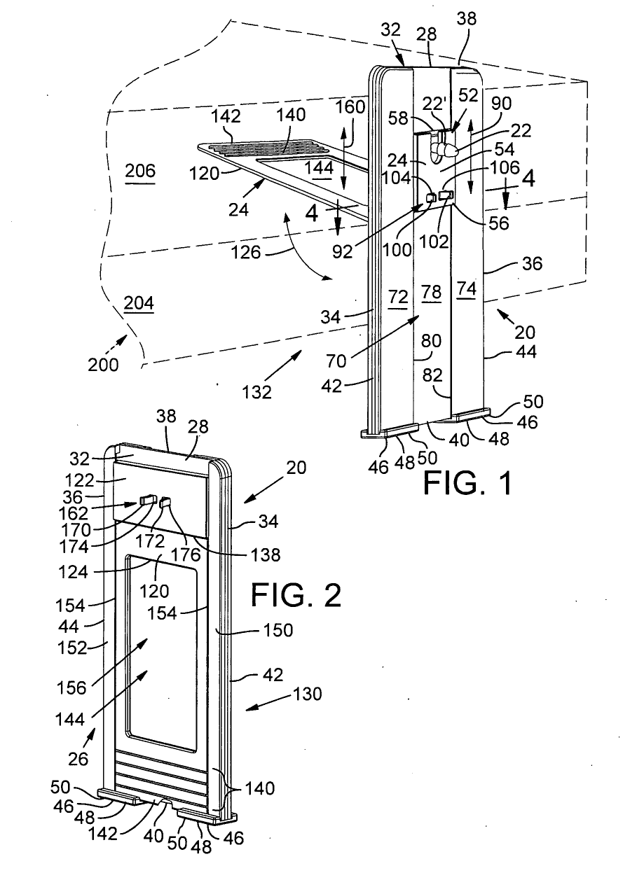

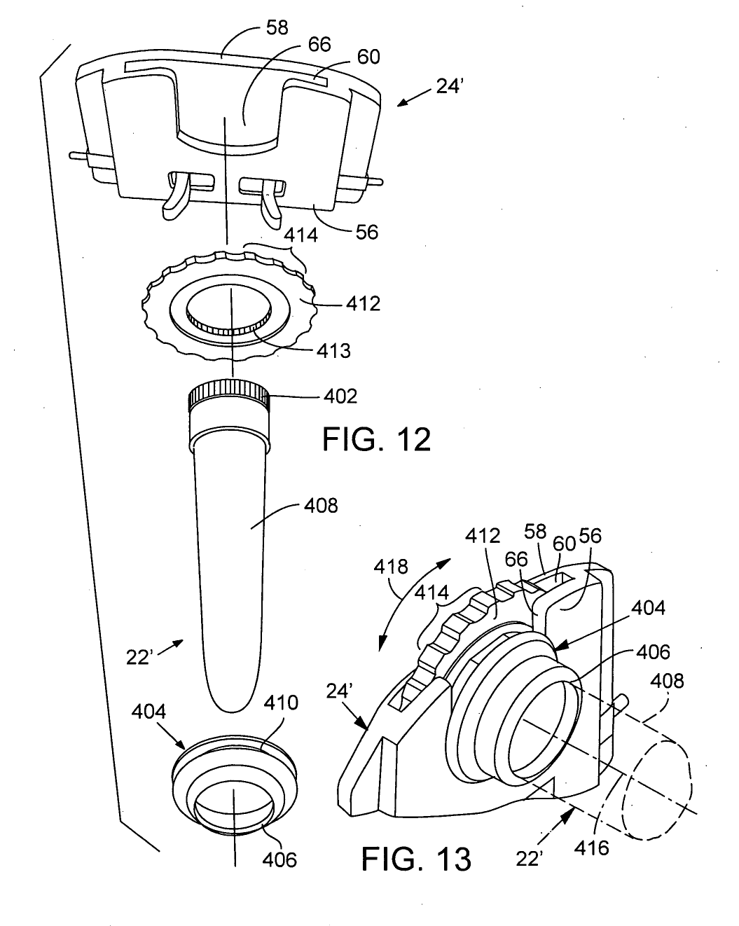

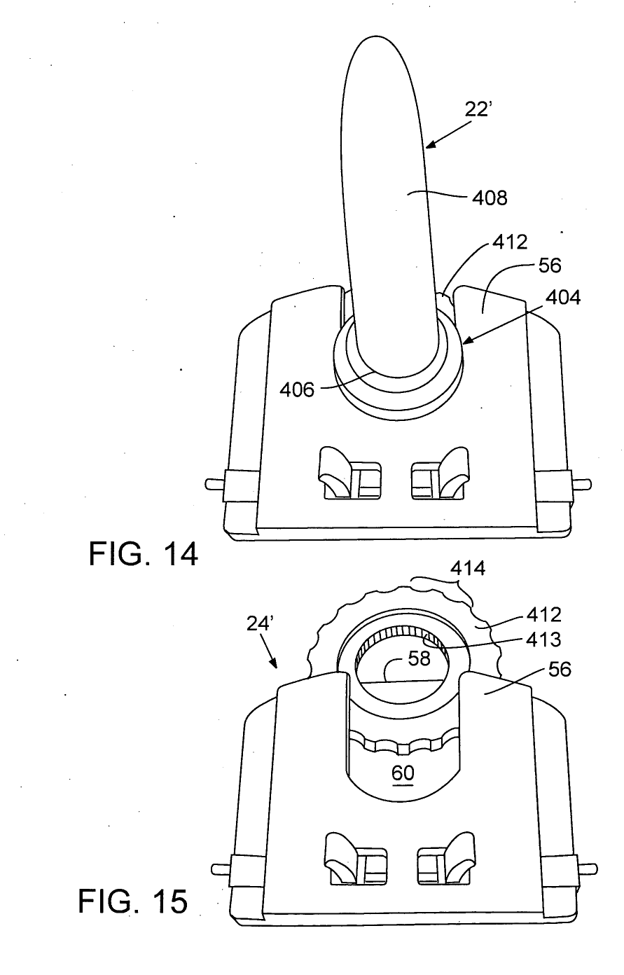

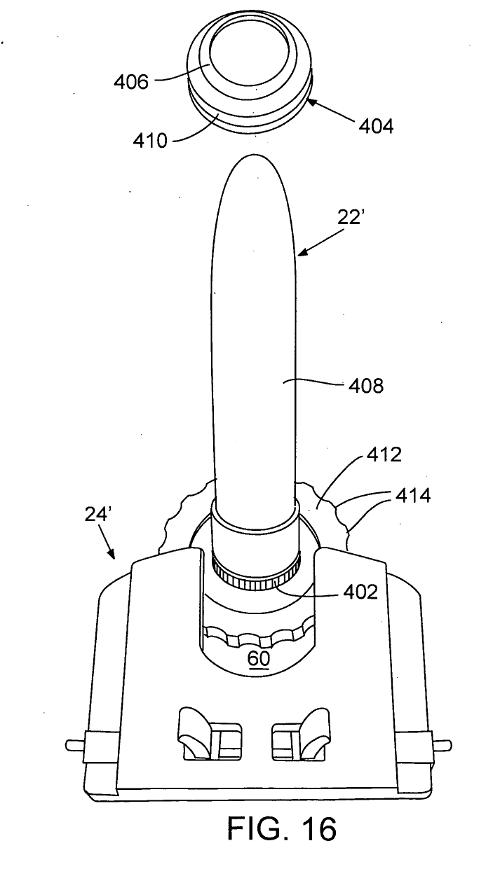

A mount 20 for a phallic device 22 is shown in FIGS. 1-7 and 12-16. The mount 20 preferably includes a height adjustable phallic device mounting portion 24 (FIGS. 1, 3, 4, & 5) and 24′ (FIGS. 12-16) and a height adjustable bed-mounting portion 26.

A. General Assembly of One Possible Phallic Device Mount

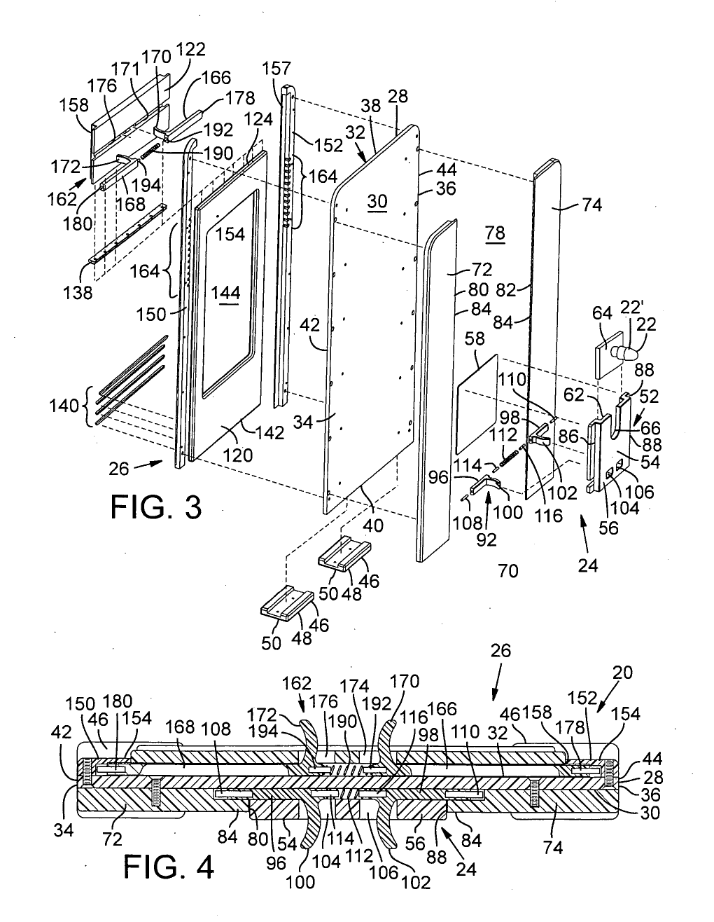

Referring to FIGS. 1 and 3, the mount 20 preferably includes a substantially planar base 28 defining a front side 30, back side 32, left side 34, right side 36, upper edge 38, lower edge 40, and left and right edges 42, 44, respectively. Feet 46, preferably having anti-slip material 48 such as rubber or the like on their lower sides 50, are secured to the lower edge 40 of the substantially planar base 28.

The phallic device mounting portion 24 includes a structure 52 for mounting a phallic device 22 such as a penis substitute, elongate sexual device, dildos, vibrators, or the like, thereto. Preferably, the phallic device mounting portion 24 includes a substantially planar front surface 54 with the phallic device 22 extending substantially perpendicular thereto. More preferably, the phallic device mounting portion 24 is substantially planar and includes a face member 56 and a substantially planar base member 58. The face member 56 is formed to define a recess 60 therein that is open along a top edge 62. The recess 60 is preferably sized to detachably receive a substantially planar base portion 64 of the phallic device 22. An opening 66 extending from the upper edge 38 of the face member 56 through the front surface 54 allows the phallic device 22 to extend therethrough.

More preferably, the phallic device 22 includes a universal mounting portion 22′ that is detachably secured to the phallic device mounting portion 24, and a plurality of different phallic devices (not shown) each of which is adapted to be installed, one at a time, onto the universal mounting portion 22′. Such universal mounting portions 22′ are known. One such universal mounting portion is disclosed in U.S. Pat. No. 5,127,396 to McAllister, the disclosure of which is hereby incorporated by reference.

The phallic device mounting portion 24 is preferably slidably secured to the front side 30 of the base 28. One possible sliding structure 70 includes positioning a left and right substantially planar member 72, 74, respectively, along the left and right sides 34, 36, respectively, of the substantially planar base 28 to define a substantially vertical channel 78 therebetween. The edges 80, 82 of the substantially planar members 72, 74, respectively, directed toward the channel 78 each include a lip 84, or flange, extending toward the channel 78. Mating recesses 86, 88 extending along the left and right sides of the phallic device mounting portion 24 slidably engage the lips 84 on the left and right substantially planar members 72, 74 thereby allowing the phallic device mounting portion 24 to slide substantially vertically in the direction of arrows 90 shown in FIG. 1.

Preferably, the phallic device mounting portion 24 includes a locking system 92 for allowing a user to select a desired height of the phallic device 22 relative to the base 28 and secure the phallic device mounting portion 24 at that height. One known locking system 92 is best shown in FIG. 3. The edges of the channel 78 include a plurality of vertically-aligned, spaced apart openings 94. The phallic device mounting portion 24 includes substantially horizontal levers 96, 98 slidably secured thereto with handle portions 100, 102, respectively, extending through parallel-aligned slots 104, 106 in the face member 56. Each lever 96, 98 includes a protrusion 108, 110, respectively, extending therefrom for matingly engaging one of the plurality of openings 94 in the edges of the channel 78. The levers 96, 98 are preferably biased to an extended position wherein the protrusions 108, 110 engage one of the openings 94. Such biasing is preferably accomplished with a spring 112 extending between the levers 96, 98 thereby urging them apart. More preferably, the spring 112 is operably secured to each lever 96, 98 by pins 114, 116 extending from each lever 96, 98 toward the spring 112 as best shown in FIG. 4.

A user adjusts the height of the phallic device mounting portion 24 by moving the handle portions 100, 102 of each lever 96, 98 towards each other, thereby disengaging the protrusions 108, 110 from their mating openings 94. The user slides the phallic device mounting portion 24 along the channel 78 to the desired height and then releases the handle portion 100, 102. The spring 112 urges the levers 96, 98 to extend the protrusions 108, 110 toward the edge of the channel 78. Accordingly, the protrusions 108, 110 are operably received within the adjacent openings 94 thereby securing the phallic device mounting portion 24 at the selected height.

The height adjustable bed mounting portion 26 preferably includes a substantially planar bed engaging portion 120 that is operably secured to a base engaging portion 122. Preferably, the base engaging portion 122 is slidably secured to the back side 32 of the base 28. More preferably, the bed engaging portion 120 is pivotally secured at a first end 124 to the base engaging portion 122 such that the bed mounting portion 26 moves with respect to the base 28 in the direction of arrows 126 (FIG. 1) to define a retracted position 130 (FIG. 2) in which the bed engaging portion 120 is positioned substantially parallel to and substantially adjacent to the base 28, and an in-use position 132 (FIG. 1) in which the bed engaging portion 120 is positioned substantially perpendicular to the base 28 as best shown in FIG. 1.

Even more preferably, the bed engaging portion 120 is pivotally secured to the base 28 with an elongate hinge 138, and anti-slip material 140, such as rubber or the like is positioned on the bed engaging portion 120 toward the opposite second end 142 of the bed engaging portion 120. In order to reduce the overall weight of the phallic device mount 20, the bed engaging portion 120 may also include a large opening 144 therein as best shown in FIG. 3.

One possible structure for slidably securing the base engaging portion 122 to the base 28 includes securing left and right rails 150, 152, respectively, each having a lip 154, or flange, extending therefrom to define a substantially vertical bed mounting portion channel 156 therebetween. Mating recesses 158 in the base engaging portion 122 operably engage the lips 154 thereby allowing the base engaging portion 122 to slide in the direction of arrows 160 (FIG. 1).

Preferably, the base engaging portion 122 includes a locking system 162 for allowing a user to select a desired height of the bed mounting portion 26 relative to the base 28 and then secure the base engaging portion 122 at that height. One known locking system 162 is best shown in FIG. 3. The rails 150, 152 include a plurality of vertically-aligned, spaced apart openings 164. The base engaging portion 122 includes substantially horizontal levers 166, 168 slidably secured thereto with handle portions 170, 172 extending through slots 174, 176 in the face of the base engaging portion 122. Each lever 166, 168 includes a protrusion 178, 180 extending therefrom for matingly engaging one of the plurality of openings 164. The levers 166, 168 are preferably biased to an extended position wherein the protrusions 178, 180 engage one of the openings 164. Such biasing is preferably accomplished with a spring 190 extending between the levers 166, 168 thereby urging them apart. More preferably, the spring 190 is operably secured to each lever 166, 168 by rods 192, 194 extending from each lever 166, 168 toward the spring 190 as best shown in FIG. 4.

A user adjusts the height of the bed mounting portion 26 by moving the handle portions 170, 172 of each lever 166, 168 towards each other, thereby disengaging the protrusions from their mating openings. The user slides the base engaging portion 122 to the desired height and then released the handle portions 170, 172. The spring 190 urges the levers 166, 168 to extend the protrusions 178, 180 toward the openings 164. Accordingly, the protrusions 178, 180 are operably received within the adjacent openings 164 thereby securing the bed mounting portion 26 at the selected height.

B. Alternative Possible Phallic Device Mount

Referring to FIGS. 12-16, an alternative possible phallic device mounting portion 24′ for securing a phallic device 22′ is disclosed. In this embodiment, the phallic device 22′ is preferably a common hand-held elongate vibrator 22′ with a rotary activation switch 402 operably secured at one distal end. Usually, this switch 402 activates and/or adjusts the vibration feature of the vibrator 22′ when a user rotates the switch about the longitudinal centerline of the phallic device 22′.

Preferably, the phallic device mounting portion 24′ includes a resilient phallic device shaft mount 404 detachably securing the phallic device 22′ to the phallic device mounting portion 24′. For example, the phallic device shaft mount 404 preferably has a collar 406 for snuggly engaging the shaft 408 of the phallic device 22′ and an opening engaging portion 410 for snuggly engaging the opening 66 within the face member 56 for the phallic device mounting portion 24′. Accordingly, the phallic device 22′ may be snugly, but detachably, secured to the phallic device mounting portion 24′ by securing the phallic device 22′ within the collar 406 and then wedging the opening engaging portion 410 within the opening 66.

The rotary switch 402 of the phallic device 22′ is preferably snuggly received within a mating opening 413 (FIG. 12) in a substantially planar ring 412. The outer diameter of the ring 412 sized so as to be received within the recess 60 formed between the face member 56 and the substantially planar base member 58 of the phallic device mounting portion 24′ when the opening engaging portion 410 of the phallic device shaft mount 404 is wedged within the opening 66 on the face member 56. If desired, the outer diameter of the ring may include notches 414 or the like to facilitate a user's ability to rotate the ring 412 as described. Accordingly, the ring 412 may be rotated about axis 416 in the direction of arrows 418 (FIG. 13), thereby allowing the rotary switch 402 on the phallic device 22′ to be activated and/or adjusted as needed during use and without the need to remove the phallic device from the mount.

C. General Assembly of Dolly

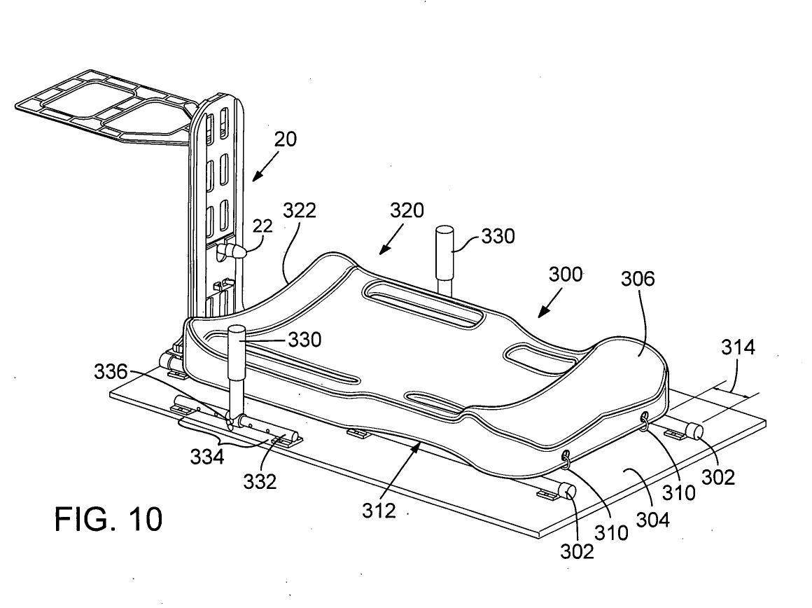

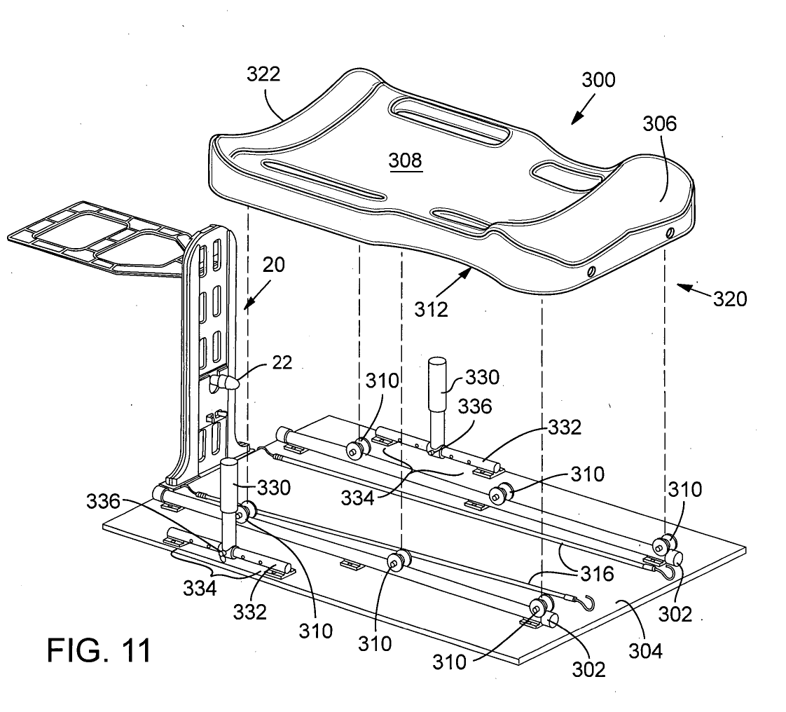

Referring to FIGS. 10 and 11, a dolly 300 for use with a phallic device 22 is disclosed. Preferably, the phallic device 22 is rigidly mounted to extend substantially horizontally above a substantially horizontal surface. Even more preferably, the dolly 300 is used with the phallic device mount 20.

The dolly 300 is preferably sized to support a user lying on their back, and the dolly 300 slides substantially along the horizontal surface thereby allowing the user to use the phallic device 22 by sliding themselves on the dolly repeatedly toward and away from the phallic device 22.

Even more preferably, the dolly 300 is biased to a neutral position and operably secured to one or more dolly rails 302 thereby maintaining proper alignment during use and offering a pleasing returning force urging the user toward the phallic device 22 during use. For example, the dolly 300 preferably includes a substantially horizontally aligned and substantially planar base 304 with a pair of parallel-aligned dolly rails 302 secured thereto. A user support 306 preferably has a top surface 308 contoured to comfortably support the user's back and head. A plurality of guide wheels 310 are pivotally secured to the backside 312 of the user support 306 to ride along the dolly rails 302 as best shown in FIG. 11. Accordingly, the user support 306 moves along the rails 302 and substantially parallel to the substantially planar base 304. Preferably, the range of movement 314 of the user support 306 along the rails 302 is about 6 to 10 inches.

Preferably, one or more resilient biasing members 316 extend between the substantially planar base 304 and user support 306 to as to bias the user support 306 toward a neutral position 320 (FIG. 10). More preferably, this neutral position is when the forward end 322 of the user support 306 is closest to the phallic device mount 30 as shown in FIG. 10. As the user support is moved along the dolly rails 302 away from the phallic device mount 20, the resilient biasing members 316 urge the user support 306 back toward the phallic device mount 20.

A pair of handles 330, one mounted on each side of the user support 306, preferably extends substantially perpendicular from the planar base 304. More preferably, the position of the handles 330 relative to the phallic device mount 20 is adjustable; say for example, by slidably securing each handle 330 to a handle rail 332 containing a plurality of spaced apart positioning holes 334 therein. A desired location of each handle 330 may be selected by aligning each handle 330 with a desired positioning hole 334 and extending a pin 336 through the handle 330 and desired positioning hole 334. Preferably, the handle 336 is positioned substantially mid-way along the range of movement 314 of the user support 306.

D. Use and Operation

A user uses the phallic device mount 20 by attaching a phallic device 22 to the phallic device-mounting portion 24 and positioning the base 28 substantially vertically next to a bed 200 as shown in FIG. 1. The user then adjusts the height of the bed mounting portion 26 so that the hinge 138 is substantially at the same height as a substantially horizontal surface 202 of the bed 200, such as the break between the box springs 204 and mattress 206. The user then moves the bed mounting portion 26 from its retracted position 130 (FIG. 2) to its in use position 132 (FIG. 1) and slides the bed engaging portion 120 between the box springs 204 and mattress 206 as shown thereby securing the base 28 to the bed 200. The weight of the mattress 206 on the bed engaging portion 120 combined with the anti-slip material 140 on the bed engaging portion 120 further secure the base 28 in place. The user then adjusts the height of the phallic device 22 by adjusting the phallic device mounting portion 24 to the desired vertical height.

Preferably, the channel 78 is sized so as to allow the phallic device 22 to be positioned substantially anywhere along the vertical length of the base 28 including being substantially close to the lower edge 40 of the base 28 and the upper edge 38 of the base 28. Accordingly, a user can use the phallic device 22 by either lying on their back on the floor adjacent to the phallic device mount 20, or, if applicable, the dolly 300, in which case the phallic device mounting portion 24 is positioned toward the lower edge 40 of the base 28, or by resting on all fours facing away from the phallic device mounting portion 24 such that their buttocks and/or vagina is aligned with the phallic device 22.

In cases where the embodiment shown in FIGS. 12-16 is installed, the user can activate the phallic device 22′ by rotating the ring 412 in the direction of arrows 418 (FIG. 13) without removing the phallic device 22′ from its mounted position on the phallic device mounting portion 24′.

E. Additional Features and Embodiments

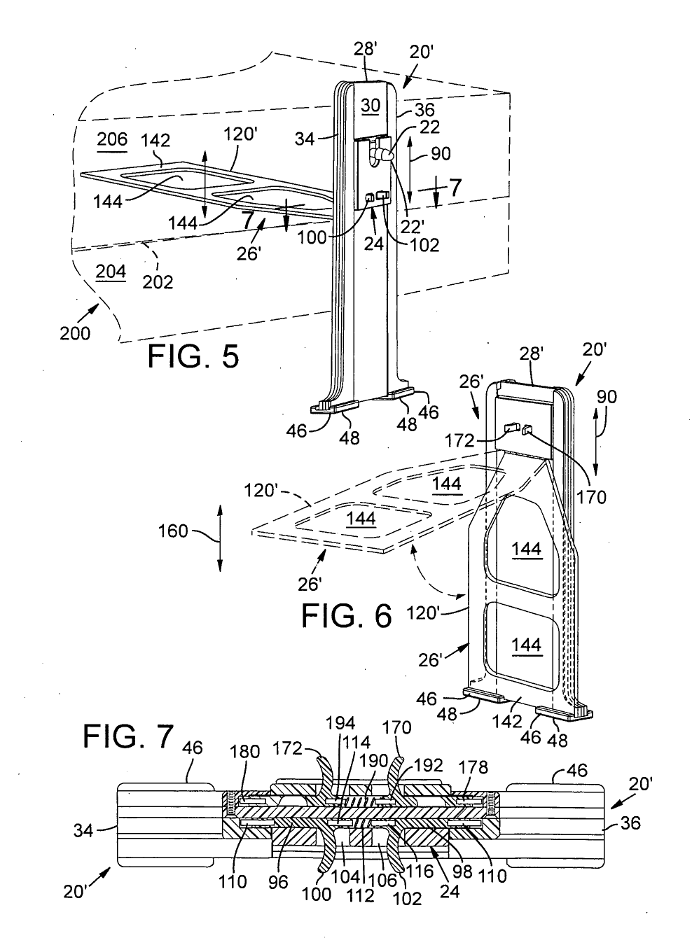

In view of the wide variety of embodiments to which the principles of the invention can be applied, it should be apparent that the detailed embodiments are illustrative only and should not be taken as limiting the scope of the invention. For example, the embodiment disclosed in FIGS. 1-4 discloses the base 28 and bed mounting portion 26 to be substantially rectangular. This allows the bed mounting portion 26 to be received within the base 28 for easy storage and concealment of the mount 20 when not in use. FIGS. 5-7 disclose different shaped structures. In this alternative embodiment, the base 28′ is much narrower thereby allowing a user's legs to pass-by on either side easily during use. Also, the bed engaging portion 120′ includes a flared outer portion 220, which preferably includes strips of anti-slip material 140 thereon, to thereby increase the surface area of the bed engaging portion 120′ and improve the rigidity and stability of the mount 20′ during use.

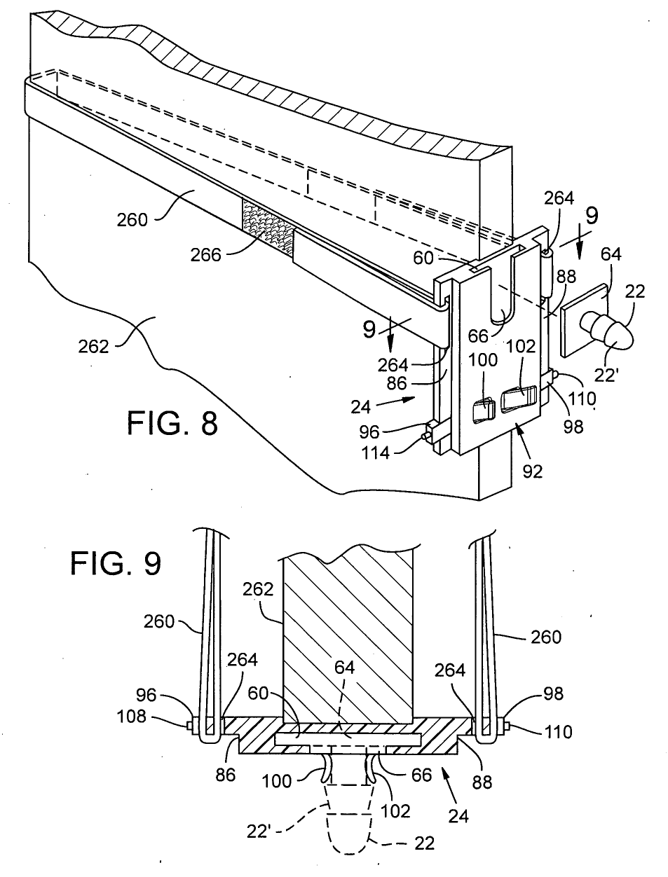

Also, as shown in FIGS. 8 & 9, the phallic device mounting portion 24 may be completely detached from the base 28 and used as a stand-alone phallic device mount. For example, a strap 260 can be secured to the phallic device 22 and wrapped around an auxiliary support structure 162, such as a door or the like, to allow a user additional opportunities to use their phallic device 22. In such case, the phallic device mount 20 can include one or more slots 264 for allowing the strap 260 to be passed therethrough. The strap 260 preferably includes securing structures such as a buckle or hook and loop material 266, thereby allowing a user to detachably secure just the phallic device mounting portion 24 to an auxiliary support structure 262.

In addition, the phallic device mount 20 of the present invention may be used in other ways as well. For example, the base 28 may be positioned substantially horizontally extending between the seats of two chairs or the like or resting flat on a substantially horizontal surface such as a bed. In such case, the bed mounting portion 26 preferably remains in its retracted position 130 (FIG. 2).

It can be appreciated that the mount 20 and/or dolly 300 can also be used to assist paraplegics, quadriplegics, and others with conditions that limit their movement, to simulate normal sexual intercourse with a partner. The adjustability of the mount 20 allows the phallic device mounting portion to be laid over, on, under or attached to a wheelchair, hospital bed or other assistive device thereby allowing such individuals to engage in a large variety of sexual experiences. In addition, the mount 20 may stand alone to allow and individual with a disfiguring condition to simulate the experience of sexual intercourse without a partner. Also, since the user support may be moved simply by a user resting on it and using their arms to move it, a paraplegic or the like can easily use the phallic device even if he or she doesn't have the physical dexterity or ability to physically thrust against it using conventional maneuvers.

In light of these additional embodiments and methods of use, the claimed invention includes all such modifications as may come within the scope of the following claims and equivalents thereto.

We claim:

1. A phallic device mount for operably securing a phallic device, the phallic device having a rotary switch toward one end, the phallic device mount comprising:
a base portion having a recess therein;
a shaft mounting portion for securing the phallic device to the phallic device mount; and,
a ring for operably engaging the rotary switch of the phallic device, said ring sized to be slidably received within said recess and having an outer portion that extends outside of the recess so that said outer portion may be grasped when the phallic device is secured to the phallic device mount, said ring pivotable about an axis when received within said recess such that the rotary switch of the phallic device may be activated by grasping the outer portion of the ring and rotating said ring about said axis.

2. The phallic device mount of claim 1, wherein said base portion is substantially planar and slidably secured to a frame.

3. The phallic device mount of claim 1, wherein the phallic device has a shaft portion and said shaft mounting portion has a collar for snuggly engaging the shaft of the phallic device.

4. The phallic device mount of claim 3, wherein said base portion has a slot and said shaft mounting portion is slidably received within said slot.

5. The phallic device mount of claim 4, wherein said shaft mounting portion has an opening engaging portion for operably engaging said slot.

6. The phallic device mount of claim 1, wherein said outer portion of said ring is circular shaped.

7. The phallic device mount of claim 6, wherein said outer portion includes notches to facilitate grasping of the outer portion.

8. The phallic device mount of claim 1, wherein said ring is substantially planar.

9. The phallic device mount of claim 1, wherein the phallic device is detachably secured to said phallic device mount.

10. The phallic device mount of claim 1, wherein said shaft mounting portion is formed of substantially resilient material.

11. The phallic device mount of claim 1, wherein said ring is formed of substantially resilient material.

12. The phallic device mount of claim 1, further including a dolly operably secured near the mount.

13. The phallic device mount of claim 1, wherein said dolly is biased to a neutral position.

14. The phallic device mount of claim 1, wherein said dolly include hand grips.
